(19) United States
(12) Patent Application Publication
(10) Pub. No.: US 2022/0304603 A1
(43) Pub. Date: Sep. 29, 2022

(54) WEARABLE DEVICE OPERABLE TO DETECT AND/OR MANAGE USER EMOTION

(71) Applicant: HAPPY HEALTH, INC., Austin, TX (US)

(72) Inventors: Dustin M. FRECKLETON, Austin, TX (US); Byron P. OLSON, Boone, IA (US); Nithin O. RAJAN, Austin, TX (US); David E. CLIFT-REAVES, Austin, TX (US)

(73) Assignee: HAPPY HEALTH, INC., Austin, TX (US)

(21) Appl. No.: 17/619,655

(22) PCT Filed: Jun. 17, 2020
(86) PCT No.: PCT/US2020/038239

§ 371 Date: Dec. 16, 2021

Related U.S. Application Data
(60) Provisional application No. 62/862,430, filed on Jun. 17, 2019.

(51) Int. Cl. A61B 5/16; A61B 5/00; A61B 5/0205; A61B 5/024; A61B 5/0533; A61B 5/08; A61B 5/33; G16H 20/70; G16H 50/20

(52) U.S. Cl. CPC A61B 5/165; A61B 5/7267; A61B 5/02055; A61B 5/02427; A61B 5/0533; A61B 5/0816; A61B 5/33; A61B 5/6802; G16H 20/70; G16H 50/20

(57) ABSTRACT

A method of monitoring an emotional state of a user including obtaining data corresponding to one or more physiological parameters of a user using one or more physiological parameters; detecting via the physiological sensors a change in at least one of the one or more physiological parameters; requesting, via a user device, an emotional state identifier corresponding to the change; and providing, via the user device, a suggested response based on the emotional state identifier.

### CROSS-REFERENCE TO RELATED APPLICATION

This application claims priority to U.S. Provisional Patent Application 62/862,430, which was filed in the U.S. Patent and Trademark Office on Jun. 17, 2019, all of which is incorporated herein by reference in its entirety for all purposes.

### FIELD

The present disclosure generally relates to a non-invasive device for monitoring physiological parameters of a user. In particular, the present disclosure relates to methods and systems for monitoring the emotional health of a user by tracking changes in one or more physiological parameters of the user.

### BACKGROUND

Numerous monitoring devices are currently available in the market configured to track various aspects of a user's health. Such devices can be capable of tracking factors such as a user's heart rate, activity throughout a defined period, steps taken throughout a defined period, wellness, and the like. Such devices can be wearable and in some examples can be integrated into garments, hats, wrist bands, watches, socks, shoes, eyeglasses, headphones, smartphones, and any other wearable item. Such devices can be configured to perform health and wellness tracking.

### BRIEF DESCRIPTION OF THE DRAWINGS

Implementations of the present technology will now be described, by way of example only, with reference to the attached figures, wherein:

FIG. 1 is a diagrammatic view of a wearable device, according to at least one instance of the present disclosure;

FIG. 2A is a diagrammatic view of a wearable device, according to at least one instance of the present disclosure;

FIG. 2B is a diagrammatic sectional view of a wearable device, according to at least one instance of the present disclosure;

FIG. 2C is a diagrammatic view of a spatially-resolved near-infrared spectroscopy (NIRS) sensor of a wearable device, according to at least one instance of the present disclosure;

FIG. 3 is a block diagram of a wearable device, according to at least one instance of the present disclosure;

FIG. 4 is a diagrammatic view of a wearable device system, according to at least one instance of the present disclosure;

FIG. 5 illustrates a block diagram of an emotional state monitoring system, according to at least one instance of the present disclosure;

FIG. 6 illustrates a diagrammatic representation illustrating a range of emotions which can be detected using the emotional monitoring device, according to at least one instance of the present disclosure;

FIG. 7A is a flow chart illustrating a method for detecting a change in the emotional state of a user, according to at least one instance of the present disclosure;

FIG. 7B is a flow chart illustrating a method for classifying the emotional state of a user based on a physiological change, according to at least one instance of the present disclosure;

FIG. 8 is a diagrammatic representation of a physiological response to emotional changes detected by respiratory rate (RR) intervals and electrodermal activity (EDA), according to at least one instance of the present disclosure;

FIG. 9 is a diagrammatic representation of a physiological response to emotional changes detected using palmer EDA, finger EDA, and heart rate, according to at least one instance of the present disclosure;

FIG. 10 is a data plot illustrating the results of a machine learning algorithm for analyzing emotion based on physiological parameters compared to hard data; and

FIG. 11 is a data plot used to determine an emotional state of a user based on previous physiological parameter data.

### DETAILED DESCRIPTION

Examples and various features and advantageous details thereof are explained more fully with reference to the exemplary, and therefore non-limiting, examples illustrated in the accompanying drawings and detailed in the following description. Descriptions of known starting materials and processes can be omitted so as not to unnecessarily obscure the disclosure in detail. It should be understood, however, that the detailed description and the specific examples, while indicating the preferred examples, are given by way of illustration only and not by way of limitation. Various substitutions, modifications, additions and/or rearrangements within the spirit and/or scope of the underlying inventive concept will become apparent to those skilled in the art from this disclosure.

### I. Terminology

As used herein, the terms “comprises,” “comprising,” “includes,” “including,” “has,” “having” or any other variation thereof, are intended to cover a non-exclusive inclusion. For example, a process, product, article, or apparatus that comprises a list of elements is not necessarily limited only those elements but can include other elements not expressly listed or inherent to such process, process, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The term substantially, as used herein, is defined to be essentially conforming to the particular dimension, shape or other word that substantially modifies, such that the component need not be exact. For example, substantially cylindrical means that the object resembles a cylinder, but can have one or more deviations from a true cylinder.

Additionally, any examples or illustrations given herein are not to be regarded in any way as restrictions on, limits to, or express definitions of, any term or terms with which they are utilized. Instead these examples or illustrations are to be regarded as being described with respect to one particular example and as illustrative only. Those of ordinary skill in the art will appreciate that any term or terms with which these examples or illustrations are utilized encompass other examples as well as implementations and adaptations thereof which can or cannot be given therewith or elsewhere in the specification and all such examples are intended to be included within the scope of that term or terms. Language designating such non-limiting examples and illustrations includes, but is not limited to: “for example,” “for instance,” “e.g.,” “In some examples,” and the like.

Although the terms first, second, etc. can be used herein to describe various elements, components, regions, layers and/or sections, these elements, components, regions, layers and/or sections should not be limited by these terms. These terms are only used to distinguish one element, component, region, layer or section from another. Thus, a first element, component, region, layer or section discussed below could be termed a second element, component, region, layer or section without departing from the teachings of the present inventive concept.

The term “physiological” as used herein (including, but not limited to, terms such as physiological sensors, physiological parameters, physiological changes, and the like) refers to an aspect/characteristic of, or appropriate to, the healthy or normal functioning of a user, specifically with respect to the user's physical or emotional health or wellbeing. Such physiological aspects can be both internal and external to the user.

As used herein, the term “machine learning algorithm” refers to a computer algorithm that automatically improves through experience. Specifically, machine learning algorithms as used herein are operable to build a mathematical model based on sample, or training, data in order to make predictions without explicit programming to do so. Various machine learning algorithms are known and can be used in accordance with the present disclosure including, but not limited to, neural network analysis, decision tree analysis, support vector machine analysis, latent Dirichlet allocation (LDA) analysis, regression analysis, Bayesian network analysis, and the like.

Additionally, the term “classifier” or “classification” as used herein refers to and identifier assigned using supervised or unsupervised learning via a machine learning algorithm to group data into predetermined categories based on some measure of inherent similarity or difference.

### II. General Architecture

The present disclosure generally relates to a portable, non-invasive emotional state monitoring device and methods for detecting a change in the emotional state of an individual based on physiological parameters. The emotional state monitoring device and methods for use thereof can find application not only in mobile devices, smartphones, and wearable devices, but also in medical applications wherein it can be critical to determine the emotional state of an individual.

The importance of mental health and emotional wellbeing has become increasingly emphasized in the recent years. A variety of changes in physiological variables can be indicative of changes in the emotional state or mental health of an individual. However, there are very few systems and methods for monitoring such changes as they relate to an individual's emotional state. Specifically, presently available systems that purport to classify the emotional state of a user provide a very general emotional state that may cover a broad range of emotions. Additionally, the algorithms used in such systems include pre-trained models created in a laboratory using laboratory conditions creating a “one-size-fits-all” type program. It has been clearly established that different people can experience the same emotions differently on a physiological level. Thus while the “one-size-fits-all” programs may be functional for a portion of the population the lack of specificity to a specific user renders the algorithm useless for other portions of the population.

The present disclosure provides systems and methods for tracking an individual's emotional state using a device to obtain data corresponding to one or more physiological parameters by detecting a change in at least one of the physiological parameters. The user device can then be used to analyze the change in physiological parameters in order to determine whether the change corresponds to a change in an emotional state. As previously mentioned, each individual experiences emotions differently. Therefore presently described user device can issue a request for the user to identify the emotional state which they are experiencing and correlate that emotional state identification to the change in physiological parameters. The systems and methods described herein can then generate a database including any number of changes in physiological states and corresponding emotional states such that the systems can build a trained mathematical model using a machine learning algorithm based on the database in order to make predictions regarding the emotional state corresponding to a future physiological change.

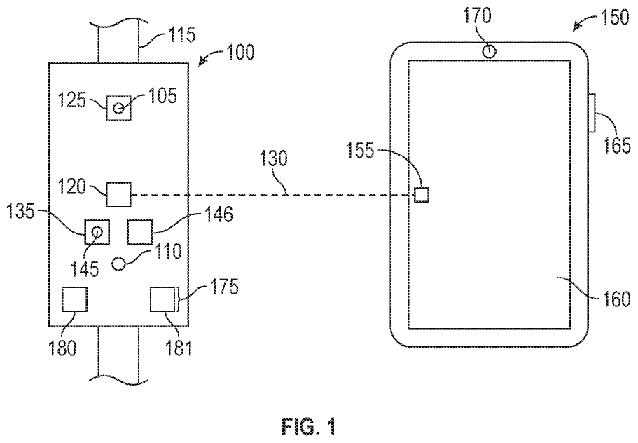

Specifically, FIG. 1 illustrates a wearable device 100, according to an instance of the present disclosure. The wearable device 100 can be operably engaged with at least a portion of a user's body. In at least one instance, the wearable device 100 can be engaged with a user wherein one or more physiological sensors of the wearable device 100 are in contact with the skin of the user. In at least one instance, the wearable device 100 can be operably engaged with the user via a band 115. In other instances, the wearable device 100 can be operably engaged with the user via a wearable clothing item including, but not limited to, a shirt, pants, shorts, compression sleeve, sock, underwear, bras, hats, helmets, bands (including headbands, wristbands, armbands, etc.), or the like. In other instances the wearable device 100 can be, but is not limited to, a watch, a ring, a band, a wristband, a necklace, a clip, a garment, or any other suitable means for contacting a user with one or more sensors disposed within the wearable device 100. In at least one instance, the wearable device can be incorporated into a medical device such as a continuous glucose monitor (CGM), an adhesive patch, or any other medical device capable of housing the sensors and communication devices described herein.

The portion of the user that the wearable device 100 is operable to be engaged with can be any of a plurality of locations including a muscle mass or tissue beds, including but not limited to, a leg, an arm, a wrist, and/or a finger of the user. In other instances, the portion of the user that the wearable device 100 is operably engaged with can include, but is not limited to, a wrist, a head, an ankle, neck, chest, abdomen, and/or other portion of the user. In at least one instance, the portion of the user that the device is attached can be the wrist for accessibility and ease of use. In other instances, the portion of the user that the device is attached can be the finger for continuous wear. The wearable device 100 can be coupled with an optional output device 150, such as a smartphone (as shown), a smartwatch, computer, mobile phone, handheld device, tablet, personal computing device, a generic electronic processing and displaying unit, cloud storage, and/or a remote data repository via a cellular network and/or wireless Internet connection (e.g. Wi-Fi).

The output device 150 can include a display 160 operable to provide a user information and/or data from one or more physiological sensors (e.g. sensor 125, 135, 175) regarding various physiological parameters. While the sensors are described herein as being one or more physiological sensors, it should be generally understood that the sensors of the wearable device disclosed herein can monitor any aspect of a user. The sensors, including the one or more physiological sensors, as described herein can include, but are not limited to, an electrodermal (EDA) sensor, a biomechanical sensor, a galvanic skin response (GSR) sensor, a photoplethysmography (PPG) sensor, an electrocardiogram (EKG), an inertial measurement sensor, an accelerometer, a gyroscope, a magnetometer, a global positioning system (GPS), a blood pressure (BP) sensor, a pulse oximetry (SpO2) sensor, a respiratory rate (RR) monitor, a temperature sensor, a humidity sensor, an audio sensor, an air quality sensor, a microphone, an environmental sensor (including but not limited to ambient noise, light, temperature, air quality, humidity, location, ultraviolet (UV) light exposure level, etc.), and/or any other sensor capable of measuring an aspect of a user and/or their environmental surroundings which may affect the user's physical and/or emotional health or wellbeing. The output device 150 can include an input control device 165 operable to allow a user to change the display 160 and/or the information and/or data displayed thereon. In at least one instance, the input control device 165 can be a button and/or other actuatable element operable to allow an input to be received by the output device 150. In other instances, the input control device 165 can be a touch sensitive input device including, but not limited to, a touch screen on a smartphone, smart watch, tablet, or the like.

The output device 150 and the wearable device 100 can be communicatively coupled 130 via a transmitter/receiver 120, 155 disposed on the wearable device 100 and the output device 150, respectively. The communicative coupling 130 can be a two-way communication pathway allowing the wearable device 100 to provide information and/or data to the output device 150 and/or the display 160 while similarly allowing the output device 150 to request information and/or data from the wearable device 100.

One or more context sensors 170 can be disposed on the output device 150 and be operable to provide data regarding a user's ambient environment (e.g. temperature, humidity, light intensity (including UV light intensity), air quality, noise level, location, etc.). In at least one instance, one or more context sensors for audio, temperature, and humidity sensing are communicatively coupled with the wearable device 100. The one or more context sensors 170 can provide comparative data for the one or more physiological sensors allowing the wearable device 100 to better understand and interpret the data measurements from the one or more physiological sensors. While the present disclosure illustrates the one or more context sensors 170 disposed on the output device 150, it is within the scope of this disclosure for the one or more context sensors to be coupled with and/or disposed on the wearable device 100, as well as coupled with and/or disposed on one or more smart home devices (e.g. smart thermostat, smart light switch, smart home hub, etc.), smart furniture items (e.g. bed sensors, chair sensors, smart refrigerators, etc.), smart bathroom fixtures (e.g. mirrors, scales, toilets having sensors, etc.), smart vehicles (e.g. cars, trucks, bicycles, e-scooters, etc.), optical sensors (including active and passive camera systems, LED and photodiode based systems, and spectroscopy systems), and the like. In at least one instance, a docking station capable of syncing and charging the wearable device 100 can include one or more context sensors communicable with the wearable device 100.

The wearable device 100 can include one or more physiological sensors such as optical sensors, thermal sensors, sweat quantification sensors, pressure sensors, electrical sensors, motion sensors, and audio sensors. Specifically, the one or more physiological sensors can include, but are not limited to, an electrodermal (EDA) sensor, a biomechanical sensor, a galvanic skin response (GSR) sensor, a photoplethysmography (PPG) sensor, an electrocardiogram (EKG), an inertial measurement sensor, an accelerometer, a gyroscope, a magnetometer, a global positioning system (GPS), a blood pressure (BP) sensor, a pulse oximetry (SpO2) sensor, a respiratory rate (RR) monitor, a temperature sensor, a humidity sensor, an audio sensor, an air quality sensor, a microphone, and combinations thereof. In at least one instance, one or more physiological sensor including, but not limited to, an EKG, an EDA, and a temperature sensor, are in contact with the user's skin. In at least one instance, one or more physiological sensors including an optical emitter and detector are in contact with the skin to measure one or more physiological parameters including PPG, BP, RR, SpO2, and combinations thereof. In at least one instance, the inertial measurement unit is in contact with the user's skin.

In at least one instance, the wearable device 100 can include a sensor 125 that is operable to determine a level of a biological and/or physiological parameter within tissue or blood vessels using near-infrared spectroscopy (NIRS). The sensor 125 can include an optical emitter 105 and/or an optical detector 110. The sensor 125 can uses one or more low-power lasers, light emitting diodes (LEDs) and/or quasi-monochromatic light sources and low-noise photodetecting electronics to determine an optical absorption. In another example, the sensor 125 can use a broad-spectrum optical source and a detector sensitive to the spectral components of light, such as a spectrometer, or a charge-coupled device (CCD) or other linear photodetector coupled with near-infrared optical filters.

The wearable device 100 can be configured to include a second sensor 135 operable to measure a photoplethysmography (PPG) of the user. The second sensor 135 can include an optical emitter 145 and/or an optical detector 146. In at least one instance, the optical system created by the optical emitter 145 and optical detector 146 can be used to quantify one or more of blood pulse volume, blood pressure, heart rate, heart rate variability, and optically opaque compounds (such as hemoglobin). The wearable device 100 can also include a third sensor 175 operable to measure electrocardiography (EKG) and/or derived systolic time intervals (STI) of the user. The third sensor 175 can include a first electrode 180 and/or a second electrode 181. The sensors 125, 135, 175 can each be a physiological sensor of the wearable device 100, collectively and/or individually. The wearable device 100 can include one or more physiological sensors including, but not limited to, sensors 125, 135, and/or 175, respectively.

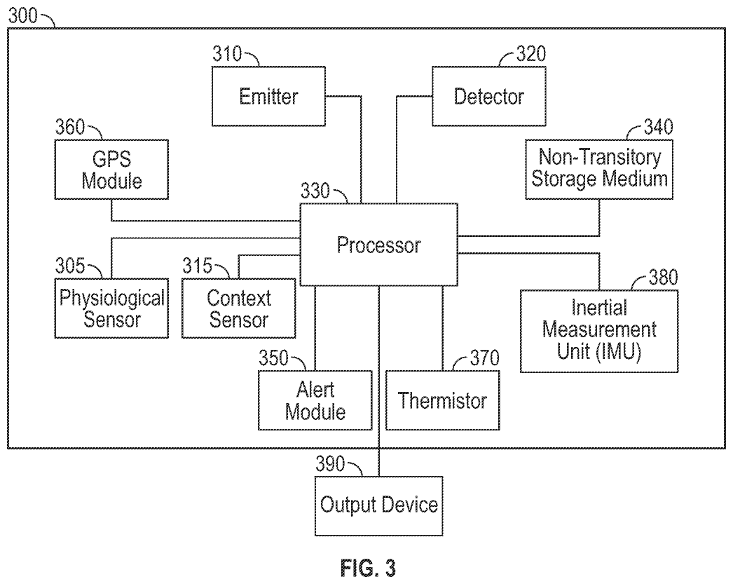

As described above, the sensors 125, 135, 175 in the wearable device 100 can measure NTRS parameters, electrocardiography, photoplethysmography, and/or derived systolic time intervals (STI) of the user. The wearable device 100 also includes a processor (shown in FIG. 3) operable to analyze data generated by one or more of the sensors 125, 135, 175 to determine a physiological response and/or physiological change of a user.

In at least one instance, the processor is operable to determine biological and/or physiological, including, but not limited to a relative percentage, a saturation level, an absolute concentration, a rate of change, an index relative to a training threshold, and a threshold. In other instance, the processor is operable to determine perfusion characteristics such as pulsatile rhythm, blood volume, vascular tone, muscle tone, and/or angiogenesis from total hemoglobin and/or water measurements.

The wearable device 100 can include a power supply, such as a battery, to supply power to one or more of the sensors 125, 135, 175 and/or other components in the wearable device 100. In at least one instance, the sensor 125 can be have a skin contact area of approximately 3.5 inches×2 inches. In other instances, the wearable device 100 can be sized to be on the user's wrist so that there is a skin contact area of approximately 1 inch×1.5 inch. In other instances, the wearable device 100 can be sized to be on the user's finger so that there is a skin contact area of approximately one quarter (¼) inch×one half (½) inch. Additionally, other dimensional skin areas are considered within the scope of this disclosure depending on the number of type of sensors operably implemented with the wearable device 100.

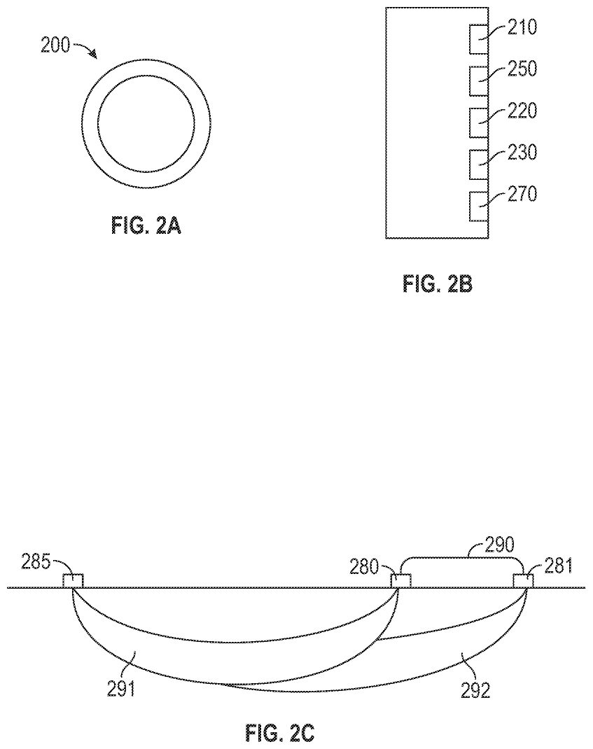

FIGS. 2A and 2B illustrate a wearable device 200 having one or more optical physiological sensors, according to at least one instance of the present disclosure. The wearable device 200 can be configured to be worn on a finger of a user. In at least one example, the wearable device 200 can be optimized to a given finger for increased accuracy. The optimization can include physiological sensor selection, arrangement, orientation, and/or shape of the wearable device 200 to ensure proper fitment. In other instances, the wearable device 200 can be optimized based on the size, gender, and/or age of the user. In still other instances, a variety of the above optimizations can be implemented for a given device.

Specifically, FIG. 2A illustrates a wearable device 200 in accordance with the present disclosure. FIG. 2B illustrates a cross-sectional view the wearable device 200, including emitters 220, 230, 250 and photodetector 210. The wearable device 200 also includes data and/or charging contacts 270. In at least one instance, the data and charging contacts 270 can be operable to electrically detect if the sensor is making contact with the skin of a user. The presence of multiple emitters 220, 230, and/or 250 on the wearable device 200 allows for spatially-resolved data gathering in real-time. In at least one instance, the wearable device 200 can be configured to determine the optical absorption of chromophores, such as water, hemoglobin in its multiple forms, including oxyhemoglobin (HbO2), deoxyhemoglobin (HHb), oxymyoglobin, deoxymyoglobin, cytochrome c, lipids, melanins, lactate, glucose, or metabolites.

FIG. 2C illustrates a spatially-resolved NIRS sensor that can be included on the non-invasive wearable device 200, according to at least one instance of the disclosure. As shown in FIG. 2C, the spatially-resolved NIRS sensor can include light emitters 280 and 281 which emit light that is scattered and partially absorbed by the tissue. Each emitter 280, 281 can be configured to emit a single wavelength of light or a single range of wavelengths. In at least one example, each emitter 280, 281 can be configured to emit at least three wavelengths of light and/or at least three ranges of wavelengths. Each emitter 280, 281 can include one or more light emitting diodes (LEDs). Each emitter 280, 281 can include a low-powered laser, LED, or a quasi-monochromatic light source, and/or any combination thereof. Each emitter 280, 281 can also include a light filter.

A fraction of the light emitted by emitters 280 and 281 can be detected by photodetector 285, as illustrated by the parabolic or “banana shaped” light arcs 291 and 292. Emitters 280, 281, are separated by a known (e.g. predetermined) distance 290 and produce a signal that is later detected at photodetector 285. The detected signal is used to estimate the effective attenuation and absorption coefficients of the underlying tissue. In at least one instance, the known distance 290 is 12 mm. In other instances, the known distance can be selected based on a variety of factors, which can include the wavelength of the light, the tissue involved, and/or the age of the user.

The wearable device 200 disclosed herein can have different numbers of emitters and photodetectors without departing from the principles of the present disclosure. Further, the emitters and photodetectors can be interchanged without departing from the principles of the present disclosure. Additionally, the wavelengths produced by the LEDs can be the same for each emitter or can be different. In at least one instance, the wearable device 200 can include additional physiological sensors as described in detail above.

In at least one instance, the wearable device 200 can be used for the monitoring of one or more physiological parameters of a user. Use of the wearable device 200 is particularly relevant in analyzing emotional changes corresponding to physiological parameters. The wearable device 200 can be configured to wirelessly measure real-time physiological parameters continuously throughout the day and/or night. The wearable device 200 can be secured to a selected muscle group, such as the leg muscles of the vastus lateralis or gastrocnemius, or any area of the user where certain physiological parameters are best measured.

FIG. 3 illustrates the components of a wearable device 300 according to at least one instance of the present disclosure. As shown in FIG. 3, the wearable device 300 can include an emitter 310 and detector 320, which can be communicatively coupled to a processor 330. The processor 330 can be communicatively coupled to a non-transitory storage medium 340. The wearable device 300 can be coupled to an output device 390.

The emitter 310 delivers light to the tissue and the detector 320 collects the optically attenuated signal that is back-scattered from the tissue. In at least one instance, the emitter 310 can be configured to emit at least three separate wavelengths of light. In another instance, the emitter 310 can be configured to emit at least three separate bands and/or ranges of wavelengths. In at least one instance, the emitter 310 can include one or more light emitting diodes (LEDs). The emitter 310 can also include a light filter. The emitter 310 can include a low-powered laser, LED, or a quasi-monochromatic light source, or any combination thereof. The emitter can emit light ranging from infrared to ultraviolet light. As indicated above, the present disclosure uses NTRS as a primary example and the other types of light can be implemented in other instances and the description as it relates to NTRS does not limit the present disclosure in any way to prevent the use of the other wavelengths of light.

The data generated by the detector 320 can be processed by the processor 330, such as a computer processor, according to instructions stored in the non-transitory storage medium 340 coupled to the processor. The processed data can be communicated to the output device 390 for storage or display to a user. The displayed processed data can be manipulated by the user using control buttons or touch screen controls on the output device 390.

The wearable device 300 can include an alert module 350 operable to generate an alert including, but not limited to, a suggested response to a detected physiological change. The processor 330 can send the alert to the output device 390 and/or the alert module 350 can send the alert directly to the output device 390. In at least one instance, the processor 330 can be operably arranged to send an alert to the output device 390 without the wearable device 300 including an alert module 350.

The alert can provide notice to a user, via a speaker or display on the output device 390, of a change in one or more physiological conditions or other parameter being monitored by the wearable device 300, or the alert can be used to provide an updated emotional indicator to a user. In at least one instance, the alert can be manifested as an auditory signal, a visual signal, a vibratory signal, or combinations thereof. In at least one instance, an alert can be sent by the processor 330 when a predetermined physiological change occurs.

In at least one instance, the wearable device 300 can include a Global Positioning System (GPS) module 360 configured to determine geographic position and tagging the physiological parameter data with location-specific information. The wearable device 300 can also include a thermistor 370 and an IMU 380. The IMU 380 can be used to measure, for example, a gait performance of a walker and/or runner and/or a pedal kinematics of a cyclist, as well as one or more physiological parameters of a user. The thermistor 370 can be used to measure, for example, temperature using either infrared systems or thermal couples. The thermistor 370 and IMU 380 can also serve as independent sensors configured to independently measure parameters of physiological threshold. The thermistor 370 and IMU 380 can also be used in further algorithms to process or filter the optical signal.

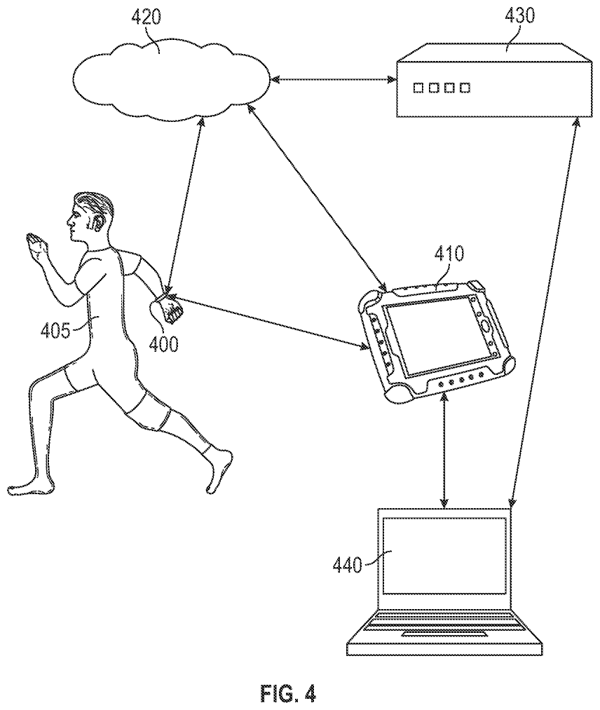

FIG. 4 illustrates an environment within which the wearable device 400 can be implemented, according to at least one instance of the present disclosure. As shown in FIG. 4, the wearable device 400 is worn by a user to determine one or more biological and/or physiological parameters. While FIG. 4 generally depicts the wearable device 400 as being worn on the wrist of a user 405, the wearable device 400 can be worn on any portion of the user suitable for monitoring biological and/or physiological parameters. The wearable device 400 can be used with an output device 410, such as a smartphone (as shown), a smart watch, computer, mobile phone, tablet, a generic electronic processing and/or displaying unit, cloud storage, and/or a remote data repository via a cellular network or wireless Internet connection.

As shown in FIG. 4, the wearable device 400 can communicatively couple with a output device 410 so that data collected by the wearable device 400 can be displayed and/or transferred to the output device 410 for communication of real-time biological and/or physiological data to the user 405. In at least one instance, an alert can be communicated from the wearable device 400 to the output device 410 so that the user 405 can be notified of a biological and/or physiological event. Communication between the wearable device 400 and the output device 410 can be via a wireless technology, such as BLUETOOTH®, infrared technology, or radio technology, and/or can be through a wire. Transfer of data between the wearable device 400 and/or the output device 410 can also be via removable storage media, such as a secure digital (SD) card. In at least one instance, a generic display unit can be substituted for the output device 410.

The wearable device 400 can communicatively couple with a personal computing device 440 and/or other device configured to store or display user-specific biological and/or physiological parameters and corresponding emotional data. The personal computing device 440 can include a desktop computer, laptop computer, palmtop, tablet, smartphone, cellphone, smart watch, or other similar device. Communication between the wearable device 400 and the personal computing device 440 can be via a wireless technology, such as BLUETOOTH®, infrared technology, or radio technology. In other instances, the communication between the wearable device 400 and the personal computing device 440 can be through a wire and/or other physical connection. Transfer of data between the wearable device 400 and the personal computing device 440 can also be via removable storage media, such as an SD card.

The output device 410 can communicate with a server 430 via a network 420, allowing transfer of user-specific biological indicator data to the server 430. The output device 410 can also communicate user-specific biological and/or physiological data to cloud-based computer services or cloud-based data clusters via the network 420. The output device 410 can also synchronize user-specific biological and/or physiological data with a personal computing device 440 or other device configured to store or display user-specific biological and/or physiological data. The output device 410 can also synchronize user-specific data with a personal computing device 440 or other device configured to both store and display user-specific data. Alternatively, the personal computing device 440 can receive data from a server 430 and/or cloud-based computing service via the network 420.

The personal computing device 440 can communicate with a server 430 via a network 420, allowing the transfer of user-specific biological and/or physiological data to the server 430. The personal computing device 440 can also communicate user-specific biological and/or physiological data to cloud-based computer services and/or cloud-based data clusters via the network 420. The personal computing device 440 can also synchronize user-specific biological and/or physiological data with the output device 410 and/or other device configured to store or display user-specific biological and/or physiological data.

The wearable device 400 can also directly communicate data via the network 420 to a server 430 or cloud-based computing and data storage service. In at least one instance, the wearable device 400 can include a GPS module configured to communicate with GPS satellites (not shown) to obtain geographic position information.

The wearable device 400 can be used by itself and/or in combination with other electronic devices and/or context sensors. The context sensors can include, but are not limited to, sensors coupled with electronic devices other than the wearable device 400 including smart devices used both inside and outside of a home. In at least one instance, the wearable device 400 can be used in combination with heart rate (HR) biosensor devices, foot pod biosensor devices, and/or power meter biosensor devices. In at least one instance, the wearable device 400 can also be used in combination with ANT+™ wireless technology and devices that use ANT+™ wireless technology. The wearable device 400 can be used to aggregate data collected by other biosensors including data collected by devices that use ANT+™ technologies. Aggregation of the biosensor data can be via a wireless technology, such as BLUETOOTH®, infrared technology, or radio technology, or can be through a wire.

The physiological parameter data aggregated by the wearable device 400 can be communicated via a network 420 to a server 430 or to cloud-based computer services or cloud-based data clusters. The aggregated data can also be communicated from the wearable device 400 to the output device 410 or personal computing device 440.

In at least one instance, the wearable device 400 can employ machine learning algorithms by comparing data collected in real-time with data for the same user previously stored on a server 430, output device 410, and/or in a cloud-based storage service. In other instances, the wearable device 400 can compare data collected in real-time with data for other users stored on the server 430 and/or in cloud based storage service. The machine learning algorithm can also be performed on or by any one of the output device 410, cloud-based computer service, server 430, and/or personal computing device 440, and/or any combination thereof.

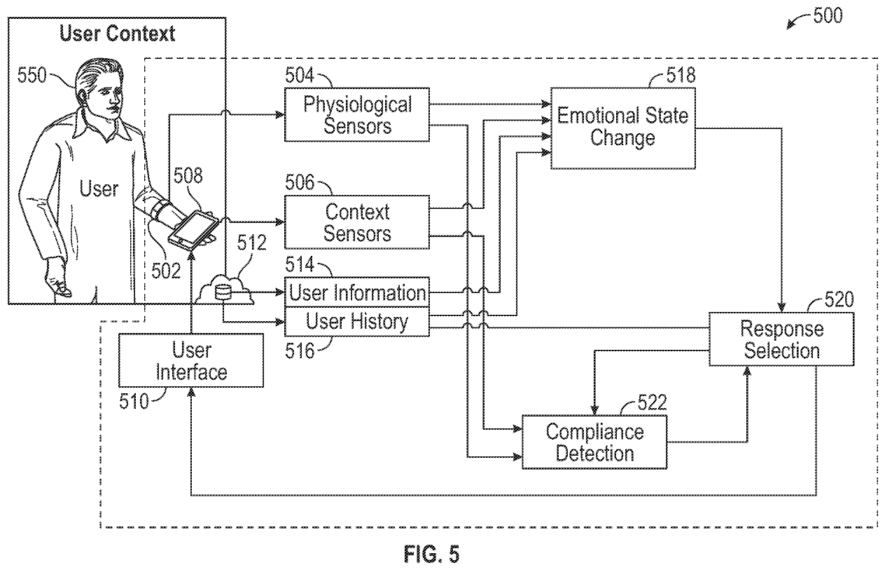

FIG. 5 illustrates a system 500 in which a wearable device 502 operable to monitor and mitigate changes in the emotional states of a user in accordance with the present disclosure. The wearable device 502 can be a watch, wristband, ring, necklace, clothing (e.g. shirt, sock, underwear, bra, compression sleeve, hats, helmets, bands (including headbands, wristbands, armbands, etc.), adhesive patch, medical device (e.g. continuous glucose monitor (CGM)), and/or combinations thereof.

The wearable device 502 which can include one or more physiological sensors 504 operably engaged with the user and operably coupled with the wearable device system 500. The one or more physiological sensors 504 can include an EDA sensor, a biomechanical sensor, a GSR senor, a PPG sensor, an EKG, an inertial measurement sensor, an accelerometer, a gyroscope, a magnetometer, a GPS, a BP sensor, a pulse oximetry (SpO2) sensor, a RR monitor, a temperature sensor, a humidity sensor, an audio sensor, an air quality sensor, a microphone, and combinations thereof. In at least one instance, the one or more physiological sensors 504 can be an optical sensor including active and/or passive camera systems operable to quantify blood pulse volume, blood pressure, heart rate, heart rate variability, and/or optically opaque compounds (such as hemoglobin, etc.). In other instances, the one or more physiological sensors 504 can include thermal systems operable to measure temperatures via infrared systems and/or thermocouples. Such thermal systems can be operable to measure body temperatures of the user 500 as well as ambient temperatures for the area surrounding the user 550. In other instances, the one or more physiological sensors 504 can include a sweat quantification system which can be a galvanic skin response and/or EDA sensor for monitoring perspiration of the user 550. In other instances, the one or more physiological sensors 504 can include a pressure system which can be implemented to monitor blood pressure. In other instances, the one or more physiological sensors 504 can include a motion system which can be implemented to monitor movement of a user 550 including, but not limited to an IU, an accelerometer, a gyroscope, a magnetometer, and/or a GPS.

The wearable device system 500 can be communicatively coupled with one or more context sensors 206 operably coupled with the wearable device 502. The one or more context sensors 506 can provide the wearable device system 500 with information about a user's ambient environment and/or location. The one or more context sensors 506 can provide ambient temperature, ambient light intensity (including UV light intensity), ambient humidity, ambient noise level, ambient air quality, and/or location. The one or more context sensors 506 can be disposed on the wearable device 502 and/or communicatively coupled with the wearable device 502. In at least one instance, the one or more context sensors 506 can include a smartphone operable to provide location information to the user. In other instances, the one or more context sensors 506 can include a smart thermostat operable to provide ambient temperature information (e.g. room temperature), a smart light switch operable to provide ambient light intensity information, a smart hub operable to provide location information within a home and noise levels, bathroom fixtures (e.g. scale, mirror, toilet with sensors, etc.), smart microphones, smart refrigerators, vehicles, and/or combinations thereof.

The wearable device system 500 can utilize the one or more context sensors 502 to appropriate, characterize, and/or provide prospective to the physiological data of the one or more physiological sensors 504.

The wearable device system 500 can further include a display 508 operable to engage with the user 500. In at least one instance, the display 508 can be a user's smartphone and can be independent of but communicatively coupled with the wearable device 502. The display 508 can provide a user interface 510 through which a user 500 interacts with the wearable device system 500.

A server 512 can be communicatively coupled with the wearable device 502 and can be operable to store user information 514 and/or user history 516. The user information 514 and/or user history 516 can include input personal information about the user (e.g., height, weight, age, gender, medical history, mental health history, etc.) and/or stored measurements obtained from the one or more physiological sensors 504 and/or the one or more context sensors 506. The server 512 can be a conventional physical server and/or a cloud-based server storage solution.

The wearable device 502 can determine an emotional state change 518 via measurements from the one or more physiological sensors 504 and/or the one or more context sensors 506. The emotional state change 518 can be indicated by changes in one or more physiological response by the user 550 (e.g. increased perspiration) while accounting for the user's environment through the one or more context sensors 506. The emotional state change 518 can have a predetermined threshold for emotional state indication in view of the user information 514 and/or user history 516 and/or collective user data obtained through a cloud storage solution.

Emotional state change can be measured and/or determined from the one or more physiological sensors by detecting a change in one or more physiological parameters measured by the physiological sensors. Examples of emotional state changes can include, but are not limited to, a change in detected heart rate (not associated with performing a physical activity), a change in breathing rate, a change in skin temperature (including an increase in perspiration and/or peripheral vasoconstriction without a corresponding change in ambient temperature as detected via the one or more context sensors 506), a change in skin conductance, a change in skin impedance, a change in blood oxygenation, a change in glucose without corresponding food ingestion, a change in skin conductivity and rate of sweat gland activation without corresponding physical activity, a change in peripheral perfusion, a change in heart rate variability, a change in blood pressure, movement deviation away from a normal pattern (e.g. pacing, excessive movement, lack of movement, excessive rest or stationary periods, etc.), a change in vocalizations (e.g. shouting, yelling, whispering, tone, word speed, etc.), and/or combinations thereof.

Upon detection of a change in emotional state above the predetermined threshold, the wearable device 502 can prompt the user to acknowledge the change by providing a series of suggested responses. In at least one instance, the response selection 520 can include several suggested tasks that can celebrate a positive mood or mitigate a negative mood of the user as measured by the one or more physiological sensors. The user 550 can select the desired response via the display 508 on a user interface 510. In other instances, the response selection can be a single option based on user information 514 and/or user history 516 as described in greater detail below.

As the user performs the selected response, the wearable device 502 can continue to monitor the user 550 via the one or more physiological sensors 504 in order to determine if the response has altered the user's emotional state as desired. In at least one instance, the user's response selection 520 can be guided or unguided. For example, the user interface 510 of the wearable device system 500 can be operable to guide the user 550 through the response selection 520 by illustrating a video, diagram, and/or other graphic. The user interface 510 can provide the user 550 with a set of instructions and/or a demonstrative video. If the user's selection is a guided response, the wearable device 502 can use a compliance detection system 522 to determine whether the user is performing the selected response properly (e.g. achieving the desired result/change in emotional state). For example, if the user has selected to perform a breathing exercise, the one or more physiological sensors 504 can monitor the user's respiratory rate to ensure they are following the guided response. In an alternative example, if the user has selected to perform an exercise, the one or more physiological sensors 504 can monitor the user's heart rate to determine if they are exercising. In another alternative example, if the user selects to take a walk, the one or more context sensors 506 can monitor ambient temperature and light to determine if the user has gone outside and the one or more physiological sensors 504 can monitor the user's location, gait, heart rate, respiratory rate, and the like to determine if the user is walking. If the compliance detection system 522 determines that the user 550 is not properly executing the selected response, the activity can be continued and/or repeated until the compliance detection 522 determines that the user 500 has successfully completed the response selection 520.

The wearable device 502 monitors the emotional state change 518 before, during, and/or after the response selection 520 activity has been performed and can determine whether the emotional state change has returned to the original state, or a state below the predetermined threshold. The wearable device system 500 can monitor, track, and learn which response selections 520 provide sufficient celebration or mitigation of an emotional change for a particular user 550 and recommend said responses more regularly. In at least one instance, if a particular response selection provides no improvement on the user's emotional state change the response can be removed from the proffered response selections. In at least one instance, the wearable device system 500 can be operable to determine different types of emotional state changes (e.g. positive emotional changes and negative emotional changes) indicated by the one or more physiological sensors 504, and recommend varying response selections 520 based on the type of emotional change detected.

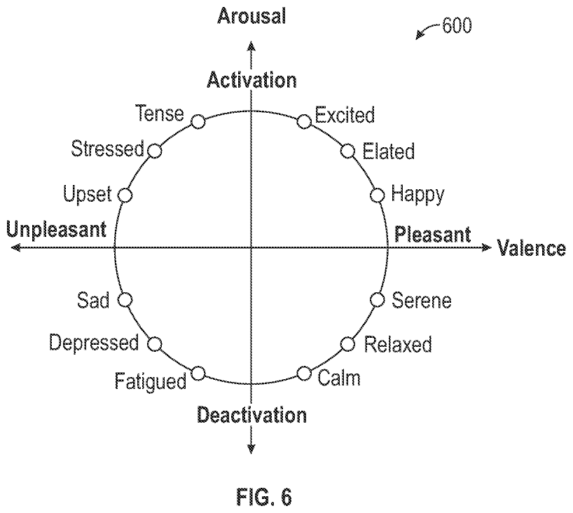

A user can experience a vast range of emotions while using the wearable device described herein. A simplified diagrammatic representation 600 illustrating a range of emotions which can be detected based on measurements of biological and/or physiological parameters using a wearable device as describe herein is provided in FIG. 6. For example, the emotional state monitoring device as described herein can be used to monitor one or more emotions including, but not limited to, afraid, alarmed, angry, tense, frustrated, annoyed, distressed, stressed, anxious, fatigued, astonished, amused, excited, elated, aroused, happy, delighted, glad, pleased, content, satisfied, serene, calm, relaxed, sleepy, tired, droopy, bored, gloomy, depressed, sad, upset, miserable, or any other emotion which the user is experiencing. As shown in FIG. 6, emotions can be generally expressed in terms of axes that represent the base components of the emotions. For example, the x- and y-axes of the diagrammatic representation 600 of FIG. 6 are valance and arousal, respectively. In at least one instance, valence refers to the intrinsic attractiveness or pleasantness (positive valence) and averseness or unpleasantness (negative valence) associated with an event, object, or situation. In at least one instance, arousal refers to the intensity of an emotion or emotional behavior. For example, whether an emotion activates or deactivates the individual feeling said emotion.

Regression techniques can be used to map physiological states to the various continuous dimensions of emotion and can subsequently be used to classify an emotion based on ordered pairs of valence and arousal. For example, the wearable device as described above can be used to monitor and detect changes in physiological parameters of a user and the systems and methods described herein can be used to correlate those changes to an emotional state of the user. For example, a variety of emotional states can be used along with a classification technique, such as neural network analysis, decision tree analysis, support vector machine analysis, latent Dirichlet allocation (LDA) analysis, regression analysis, Bayesian network analysis, and the like to create an emotional state classifier.

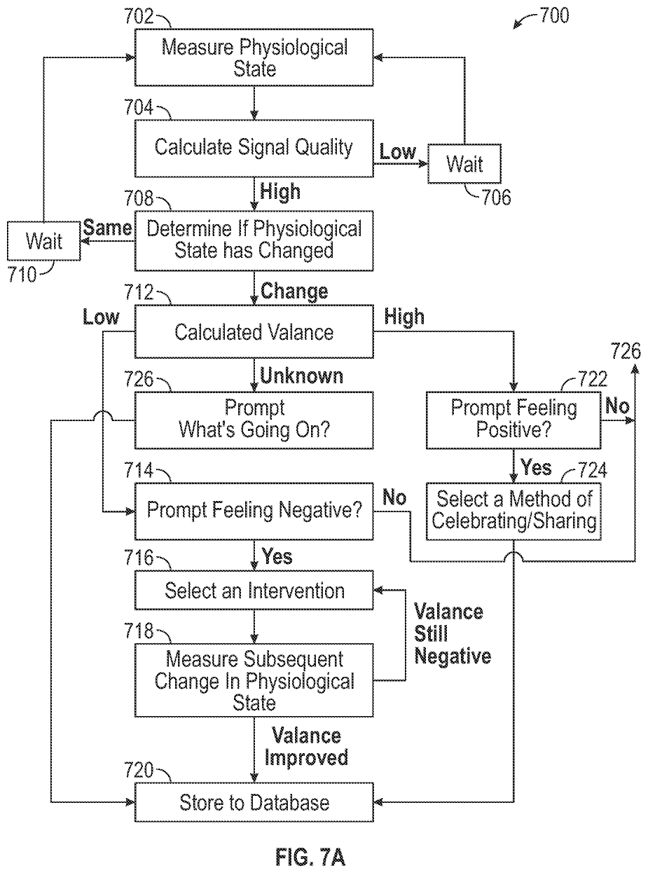

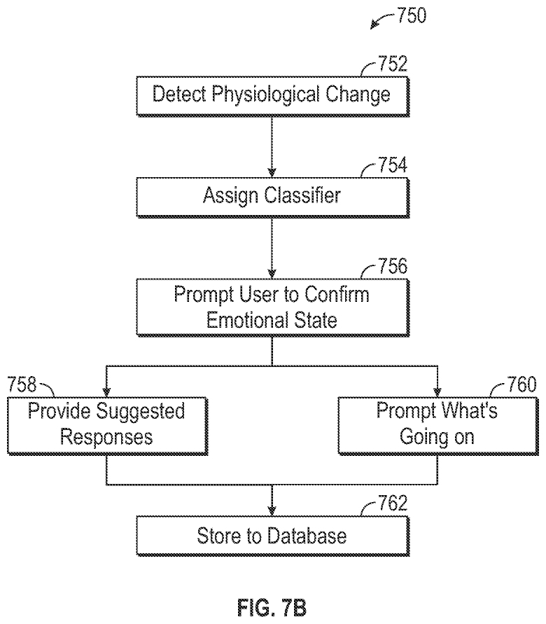

An exemplary method 700 for monitoring a user's emotional state using the wearable device described above is provided in FIG. 7A. The method 700 can begin at block 702 where one or more physiological parameters are measured to determine a physiological state of the user. The physiological parameters can be measured using one or more of the physiological sensors as described in detail above. In at least one instance, the physiological state determined using physiological parameters can include, but is not limited to, heart rate (HR), heart rate variability (HRV), breathing rate (BR), blood pressure (BP), electrodermal (EDA), vocal stress, physical movement, and combinations thereof.

At block 704, one or more processors communicable with the wearable device can determine whether the signal quality of the physiological state is sufficient for an accurate measurement to be taken. In at least one instance, quality algorithms can be used to determine if the measurements or of sufficient quality to attempt a screening measurement. If it is determined that the quality is insufficient (e.g. too low) to obtain an accurate reading, the method 700 can proceed to block 706 where the wearable device waits until a signal having a sufficient quality is detected.

If, at block 704 the signal is determined to be sufficient (e.g. high quality), then the method 700 can proceed to block 708. At block 708, the method can determine if the physiological state has changed, based at least in part on a change in one or more physiological parameters measured by the physiological and context sensors associated with the wearable device. In determining whether a change has occurred, the present physiological state is compared to the last known physiological state. For example, the last physiological state can be the most recent physiological state that provided sufficient signal quality for measurement. If the present physiological state is the same as the last known physiological state, the method 700 can proceed to block 710, where the method waits until another measurement is taken. In at least one instance, the wait period at block 710 can be a predetermined period of time. In an alternative instance, the wearable device can continuously measure physiological states as long as the wearable device is engaged with the user. In such instance, the wait period at block 710 can be negligible. In at least one instance, the method 700 can adjust sensitivity to the change in physiological state based on the user. For example, if the user frequently experiences a brief negative mood that is mitigated without any intervention, the method 700 can wait a predetermined period of time before indicating a negative mood until after the brief period has passed. The present physiological state is recorded and used as the last known physiological state when the method 700 repeats.

In the alternative, if a change in physiological state is detected at block 708, the method can proceed to block 712. In at least one instance, the change in physiological state can be required to exceed a predetermined threshold, such that the method 700 is only triggered when a “significant” change in physiological state is detected. At block 712, the valance and arousal of the user is calculated to determine an anticipated emotional state for the user. As discussed in detail above, emotions can be present in ranges. When an individual first beings using the wearable device, the method proceeds based on an initial determination of a positive or negative emotion based on a generalized view of which range of emotions trend towards positive or negative. As the individual continues to use the device, a model is created as described below which is tailored to the individual's emotional experience.

If the valance and arousal are determined to be low, the method 700 can proceed to block 714. At block 714, the wearable device issues a prompt to the user asking whether the user is feeling negative. If the user confirms that they are experiencing a negative emotion, the method proceeds to block 716. At block 716, the user is prompted to select an intervention or mitigation designed to increase their valance and arousal. In at least one instance, the interventions include one or more suggested responses including, but not limited to, performing a breathing exercise, performing physical exercise (going for a walk, a jog, attending an exercise class, etc.), listening to music, turning down lights, taking a nap, talking to someone (calling a friend, seeking professional help, visiting a relative, etc.), writing in a journal, executing a hobby (painting, practicing an instrument, knitting, etc.), diffusing a calming oil, moving locations (going outside or inside, going to a friend's house, going to a park, etc.), meditating for a predetermined period of time, watching a video, participating in psychotherapy (including cognitive behavioral therapy (CBT)), engaging in social interaction, performing an act of kindness, or any other process which may increase serotonin production or provide a calming sensation. The suggested responses can be provided based at least in part on the user's personality type, the time of day, the last known intervention or exercise performed by the user, a user preference, a geographic location, a complete physiological state of the user, and combinations thereof. For example, while the initial use period will provide generalized suggested responses, as the individual continues to use the emotional state monitoring device the suggested responses can become more tailored to the individual. In at least one instance, the suggested responses can be determined at least in part by how effective the response has been for others having a similar emotional state in similar situations.

At block 718, the physiological state of the user is measured for a subsequent change. Specifically, a baseline physiological state for the individual is recorded prior to beginning the intervention selected from the suggested responses. The wearable device as described herein can continuously monitor the individual throughout the duration of the intervention to determine how the physiological state of the user changes as they perform or subsequent to the intervention task. In at least one instance, the intervention can be guided or unguided. Where the intervention is guided, the wearable device can track the performance of the intervention using the physiological and context sensors as described above to ensure compliance with the intervention. For example, if the individual selects to perform a guided breathing exercise, the wearable device can track the individuals' breath during the guided intervention to determine if the breathing exercise was performed as requested. Where the intervention is unguided, the wearable device can track all physiological changes as the individual performs their selected intervention.

After the intervention is completed, the physiological state of the individual is assessed. If the valance and arousal of the individual has increased, the method can proceed to block 720 where the information is stored to a database. The database can include at least data corresponding to the initial change in physiological state that triggered the method, the user's selected intervention, and the subsequent change in physiological state caused by performing the intervention. In at least one instance, the database can also include information relating to user information and user history as described above. As described above, the information stored in the database can be used to train a model to tailor the physiological state analysis and suggested responses to what best suits the individual.

In the alternative, if the individual's valance and arousal have not increased after performing the intervention, the method 700 can return to block 716 where an alternative intervention is suggested. In at least one instance, information relating to an unsuccessful intervention is also stored to the database. Such information can be used to narrow the suggested responses in the future to those known to be effective for the individual. The method 700 can repeat until the valance and arousal of the individual has increased.

In the alternative, if the user indicates that they are not experiencing a negative emotion at block 714, the method 700 proceeds to block 726 where the user is prompted to indicate what is going on. The method 700 with respect to block 726 is explained in greater detail below.

Referring back to block 712, if the calculated valance and arousal is determined to be high, the method 700 can proceed to block 722. At block 722, the wearable device issues a prompt to the user asking whether the user is feeling positive. If the user confirms that they are experiencing a positive emotion, the method proceeds to block 724 where the user is prompted to celebrate the positive emotion. In at least one instance, the celebrations include one or more suggested responses including, but not limited to, writing in a journal, sharing a positive message (posting on social media, etc.), performing physical exercise (going for a walk, a jog, attending an exercise class, etc.), listening to music, talking to someone (calling a friend, visiting a relative, etc.), executing a hobby (painting, practicing an instrument, knitting, etc.), moving locations (going outside or inside, going to a friend's house, going to a park, etc.), meditating for a predetermined period of time, watching a video, participating in psychotherapy (including CBT), engaging in social interaction, performing an act of kindness, or any other process which can validate and/or increase the positive emotion. As discussed above, the suggested responses can be provided based at least in part on the user's personality type, the time of day, the last known celebration or exercise performed by the user, a user preference, a geographic location, a complete physiological state of the user, and combinations thereof. For example, while the initial use period will provide generalized suggested responses, as the individual continues to use the emotional state monitoring device the suggested responses can become more tailored to the individual. In at least one instance, the suggested responses can be determined at least in part by how effective the response has been for others having a similar emotional state in similar situations.

As described above, the method 700 can monitor one or more physiological parameters to determine if a subsequent change in emotional state occurs during or subsequent to the celebration. The celebration can be guided or unguided. The wearable device can monitor the physiological state of the user as the celebration is performed to determine what effect the celebration had on the physiological state of the user. The method 700 can then proceed to block 720 and store the data to the database. The data can include at least the initial change in physiological state that triggered the method, the user's selected intervention, and the subsequent change in physiological state caused by performing the intervention. In at least one instance, the database can also include information relating to user information and user history as described above. Such data is used to train the model to tailor the suggested responses to the individual using the wearable device.

In the alternative, if the user indicates that they are not experiencing a positive emotion at block 722, the method 700 proceeds to block 726 where the user is prompted to indicate what is going on.

Referring back to block 712, if the calculated valance and arousal does not indicate a distinctly positive or negative emotion, the method proceeds to block 726 where the user is prompted to indicated what is going on. In at least one instance, the prompt can include a request for the user to provide an emotional state identifier that they believe corresponds to their current emotional state. For example, the emotional state identifier can include, but is not limited to, afraid, alarmed, angry, tense, frustrated, annoyed, distressed, stressed, anxious, fatigued, astonished, amused, excited, elated, aroused, happy, delighted, glad, pleased, content, satisfied, serene, calm, relaxed, sleepy, tired, droopy, bored, gloomy, depressed, sad, upset, miserable, or any other emotion which the user is experiencing. In at least one instance, a selectable list of emotional state identifiers may be provided to the user via the wearable device or output device as described herein. In other instances, the user may input any emotional state identifier they desire using the wearable device or output device as described herein. As indicated above, individuals can experience emotions differently based on their background. As such, individual users can experience different physiological responses to the same stimuli. The method 700 can then proceed to block 720 where the data is stored to the database. The data can include at least the initial change in physiological state that triggered the method and the user's emotional state identifier. Such information can be used in further methods 700 to determine how the individual reacts physiologically to certain emotions and allow for more helpful suggested responses when the user experiences future positive and negative emotions. For example, a first individual may experience with a rise in heart rate, drop in skin temperature, and rise in skin conductivity when experiencing a negative emotion. On the contrary, a second individual may experience an increase in skin temperature and an increase in respiratory rate when experiencing the same negative emotion. As such, the model must be tailored, as described above, to the specific user's physiological changes in response to their emotions. The customization of the model takes place over an extended period of time as the user interacts with the wearable device.

In at least one instance, if the user identifies the emotion as a negative or positive emotion the method 700 can proceed to block 714 or block 722, respectively, and proceed as indicated above.

The wearable device as described herein can be used with one or more machine learning algorithms and techniques that allow the wearable device to classify a user's emotional state based on a detected change in one or more physiological parameters. For example, FIG. 7B illustrates a method 750 for verifying the classification assigned using machine learning algorithms. The method 750 can begin at block 752 where a physiological change is detected in the user. As described above, the change can include a variation in one or more physiological parameters monitored using the physiological and context sensors described herein.

Once detected, one or more processors associated with the wearable device can access the database described above. In at least one instance, the one or more processors can to review information stored on the database including, but not limited to, data relating to initial change in physiological state that triggered the method, the user's selected intervention, the subsequent change in physiological state caused by performing the intervention, user information, and user history. The one or more processors can use the information stored in the database to train a model to classify variations in the physiological parameters of the user as corresponding to specific emotional states. At block 754, the one or more processors uses this data to assign a classifier to the detected physiological change, the classifier identifying the presumed emotional state of the user. At block 756, the method 750 prompts the user to confirm whether the emotional state classification is accurate.

If they user confirms that the assigned classifier is an accurate description of their present emotional state, the method 750 can proceed to block 758 where one or more suggested responses are provided to the user. The suggested responses can include, but are not limited to, interventions, mitigations, and celebrations as described above. In accordance with the method 700, the one or more physiological sensors can monitor the user's participation in the suggested response and record subsequent physiological changes. The method 750 then stores all data gathered including, but not limited to, the detected physiological change, the assigned classifier, the selected suggested response, and any subsequent physiological changes to a database at block 762.

In the alternative, if at block 756 the user indicates that the assigned classifier is not an accurate description of their present emotional state, the method 750 can proceed to block 760 where the user is prompted to indicate what is going on. For example, the user can be prompted to provide an accurate emotional state identifier corresponding to their current emotional state. Once the emotional state of the user is properly identified, the method can revert to block 756 and proceed through the end of the method 750. Such process can proceed as described with respect to block 726 of FIG. 7A. The method 750 can then proceed to block 762 where the information is stored to a database. The information stored can include, but is not limited to the detected physiological change, the assigned classifier, the correct emotional state identifier, the selected suggested response, and any subsequent physiological changes.

In at least one instance, the database described in block 762 is the same database as that described with respect to block 720 of FIG. 7A. In other instances, the database described with respect to block 762 can be a secondary database used to further train the model based on the individual user's preferences and reactions.

### EXAMPLES

The following examples are provided to illustrate the subject matter of the present disclosure. The examples are not intended to limit the scope of the present disclosure and should not be so interpreted.

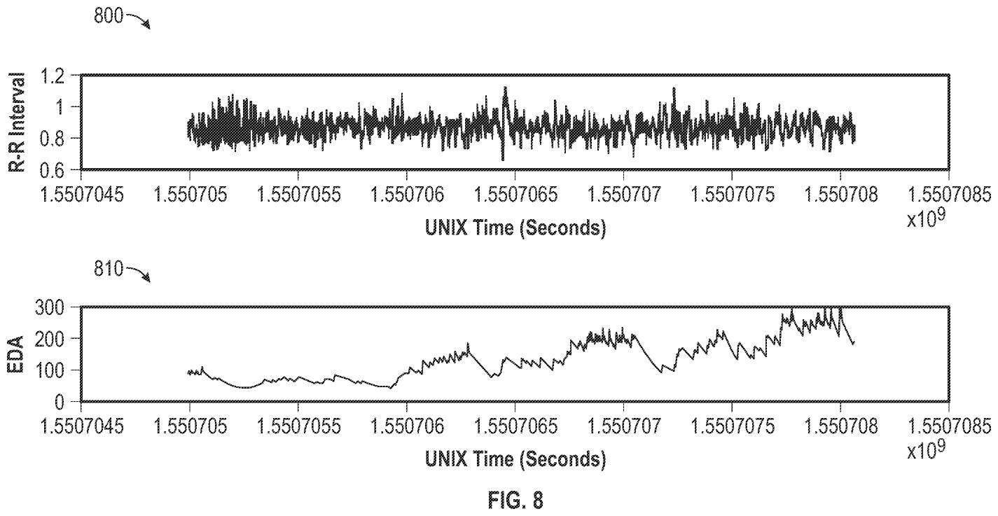

A first experiment involved the physiological changes of a subject wearing the above described wearable device was collected. The data corresponding to the change in various physiological states as time progressed are illustrated in graphs 800 and 810 of FIG. 8. Specifically, the changes in the emotional state of the subject were induced over a period of time by having the subject watch a series of music videos with high emotional valence. The change in physiological parameters were monitored so that they could later be related back to the emotional state which the user was experiencing.

For example, graph 800 illustrates a change in respiratory rate (RR) intervals over a period of time (measured in seconds) as the subject watched the series of music videos. The wearable device simultaneously tracked additional physiological parameters using an electrodermal sensor (EDA) as the subject viewed the videos, the results are provided in graph 810. The EDA sensor can be used to measure Galvanic skin response. Each spike in the physiological parameter measured by the EDA sensor is associated with the subject watching a new video, followed by a washout period which allowed the subjects physiological state to return to a more baseline level.

The data was analyzed and it was determined that the wearable device provided an indication of the user's emotional state having 80.31% accuracy. For example, Table 1, below, illustrates an indicates a sample classification of two dimensional emotional state.

TABLE 1

| | Predicted 1 | Predicted 2 | Predicted 3 | Predicted 4 |
|---|---|---|---|---|
| 1 - Happiness | 71 | 0 | 4 | 5 |
| 2 - Love | 4 | 64 | 6 | 6 |
| 2 - Sadness | 9 | 6 | 59 | 6 |
| 4 - Disgust | 5 | 5 | 7 | 63 |

A partial least squares (PLS) model allows the data obtained from the wearable device to be collected and regress the variables against the precise valence and arousal of each video the subject was enticed to watch. Such approach allows for a more nuanced view of the repose of the subject to the video.

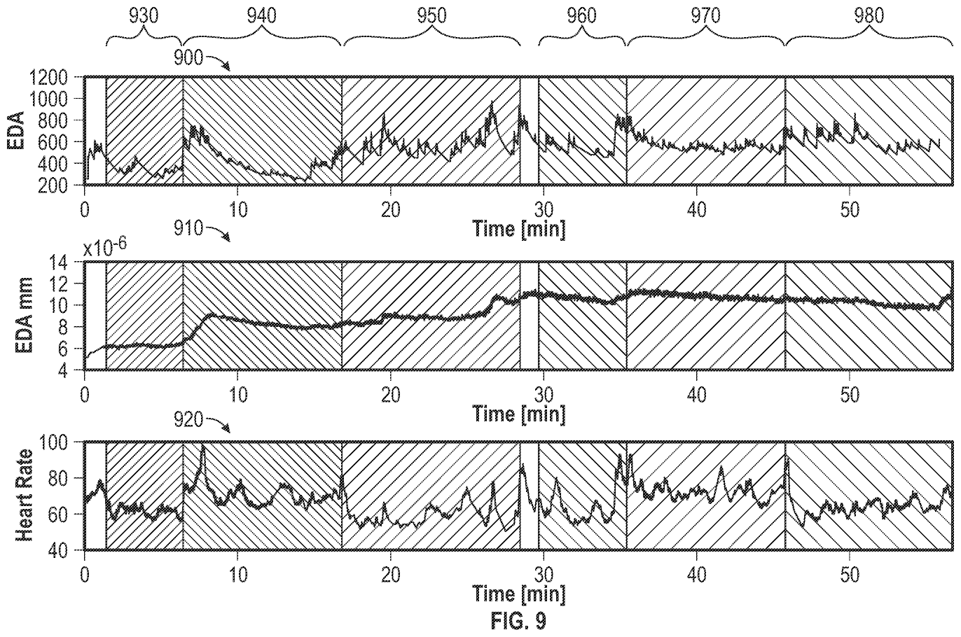

A second experiment was performed wherein the physiological changes of a subject over time were evaluated in response to happy and sad emotional content. The recorded changes in physiological parameters corresponding to the second experiment is illustrated in graphs 900, 910, and 920 of FIG. 9. Specifically, graph 900 illustrates changes in a physiological parameter as measured by a palm EDA sensor, graph 910 illustrates changes in a physiological parameter as measured by a finger EDA sensor, and graph 920 illustrates changes in a physiological parameter as measured by a heart rate sensor. Regions 930 and 960 of the graphs 900, 910, 920 are wash out periods before and after emotionally charged periods where the subject was not exposed to any particular emotional stimuli.

Regions 940 and 950 indicate periods of time when the subject thought about happy or positive times and watched videos containing happy or positive scenes, respectively. Similarly, regions 970 and 980 indicate periods of times when the subject thought about sad or negative times and watched videos that contained sad or negative scenes, respectively. As illustrated in the graphs 900, 910, 920, the emotionally charged activities cause a change in the physiological parameters of the subject. These changes can them be correlated to the subject's positive and negative emotions, such that in the future if the wearable device detects one of the physiological parameter changes indicated in FIG. 9 the model can provide the corresponding emotion.

A third experiment was performed, wherein several subjects were instrumented with an EDA sensor and a HR sensor. A baseline measurement was taken of each subject to establish normal values for various physiological parameters including, but not limited to, RMSSD, nLF, SD1, HR, and combinations thereof. After the baseline was established, a series of videos were played for the subjects and their physiological parameters were monitored for changes. Each video was a minute long and there was a ten-second break between each of the videos.

Changes in heart rate variability and EDA values were calculated during each of the videos. In addition to the changes between videos, values calculated for each of the videos was additionally compared to the baseline values originally collected. The calculated values were then used to predict the emotional state that the subjects experienced.

Separately, each of the videos was classified as corresponding to a specific emotional state by a large number of subjects. Each video was assigned a class based on the average valance and arousal indicated by the subjects. A machine learning algorithm was used to create a classifier based on the actual emotional states provided. Specifically, the classifier took the heart rate and EDA sensor data and fit a multi-class linear discriminant analysis (LDA) to learn the class of each video. Thus by examining the HR and EDA recorded during a video, and applying the LDA, a predicted emotional value (e.g. the valance-arousal quadrant) of the video.

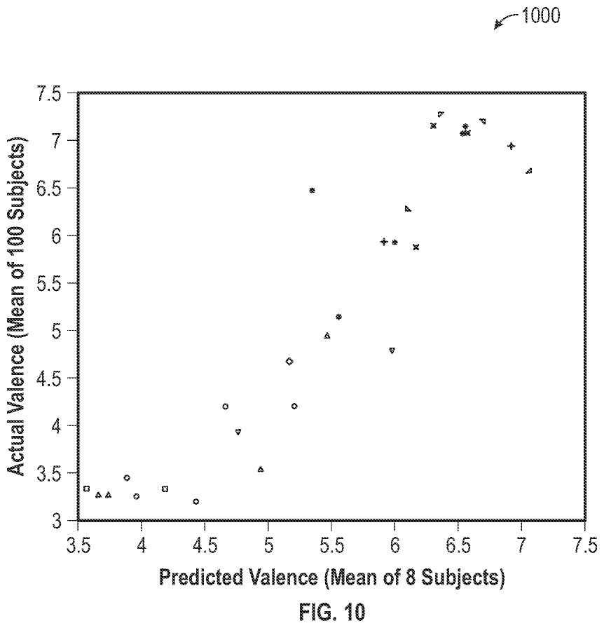

FIG. 10 illustrates a graph 1000 showing the high agreement between the prediction by the presently disclosed wearable device and the emotional valance that the videos induced in a range of subjects. Each shape in the graph 1000 illustrates a distinct video that has a known emotional valance and arousal associated with it.

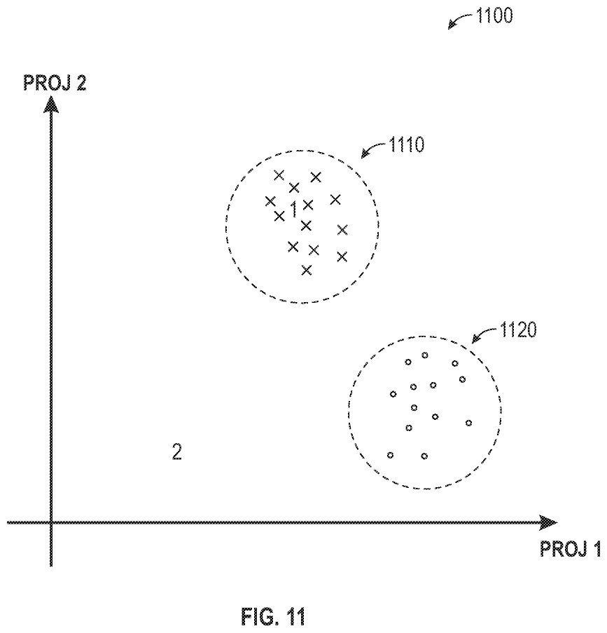

For example, FIG. 11 further illustrates a graph 1100 that depicts a high dimensional space in which all of the relevant physiological and context variables are represented in accordance with the model described herein. The space of graph 1100 is observed via two projections (Proj 1 and Proj 2). When data point 1 is observed, the model assumes the subject is experiencing an emotion associated with region 1110 because data point 1 fits squarely within the region 1110. Therefore, the wearable device would not need to ask the user what emotion they were experiencing because it was successfully predicted. The method described above can proceed directly to encouraging the user to celebrate the positive emotion.

On the contrary, when data point 2 is observed, the model would not be able to immediately recognize the emotion experienced by the user because the data point does not fall within known emotional regions 1110 or 1120. With respect to the method described above, the wearable device would prompt the user to provide an emotional state identifier. The model would then learn to classify data point 2 with the corresponding emotional state identifier provided by the user.

The embodiments shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the embodiments described above may be modified within the scope of the appended claims.

Numerous examples are provided herein to enhance understanding of the present disclosure. A specific set of statements are provided as follows.

Statement 1: A method of monitoring an emotional state, the method comprising obtaining, via one or more physiological sensors, data corresponding to one or more physiological parameters of a user; detecting, via at least one of the one or more physiological sensors, a change in at least one of the one or more physiological parameters; requesting, via a user device, an emotional state identifier corresponding to the change; and providing, via the user device, a suggested response based on the emotional state identifier.

Statement 2: A method in accordance with Statement 1, further comprising generating, via one or more processors communicatively coupled with the user device, a database comprising the change in the at least one of the one or more physiological parameters and corresponding emotional state identifier; and providing a classifier for each emotional state identifier, the classifier corresponding to an emotion experienced by the user.

Statement 3: A method in accordance with Statement 1 or Statement 2, further comprising training the one or more processors to assign the classifier to a subsequent change in at least one of the one or more physiological parameters based at least in part on the database; and requesting, via the user device, a confirmation that the classifier is accurate.

Statement 4: A method in accordance with any of Statements 1-3, wherein when the request for confirmation is received: updating the database based on the confirmation.

Statement 5: A method in accordance with any of Statements 1-4, wherein when the request for confirmation is denied, requesting, via the user device, the emotional state identifier for the subsequent change; and updating the database based on the emotional state identifier provided.

Statement 6: A method in accordance with any of Statements 1-5, further comprising monitoring, via the one or more physiological sensors, the one or more physiological parameters to determine if the user complied with the suggested response; determining, via the one or more processors, whether compliance with the suggested response altered the classifier corresponding to the user's emotion; and storing, in a second database, the alteration the suggested response had on the user's emotion.

Statement 7: A method in accordance with any of Statements 1-6, wherein the one or more physiological sensors comprises an electrodermal (EDA) sensor, a biomechanical sensor, a galvanic skin response (GSR) sensor, a photoplethysmography (PPG) sensor, an electrocardiogram (EKG) sensor, an inertial measurement sensor, an accelerometer, a gyroscope, a blood pressure sensor, a pulse oximetry (SpO2) sensor, a respiratory rate monitor, a temperature sensor, a humidity sensor, an audio sensor, an air quality sensor, and combinations thereof.

Statement 8: A method in accordance with any of Statements 1-7, wherein at least one of the one or more physiological sensors are coupled with the user device.

Statement 9: A method in accordance with any of Statements 1-8, wherein the user device is a wearable device selected from the group comprising a watch, a wrist band, a ring, a necklace, a clothing item, an adhesive patch, a medical device, a hat, a helmet, a band, and combinations thereof.

Statement 10: A method in accordance with any of Statements 1-9, wherein the suggested response comprises a positive emotion reinforcement, a negative emotion mitigation, an emotional state acknowledgement, and combinations thereof.

Statement 11: A method in accordance with any of Statements 1-10, receiving at the one or more processors data from one or more context sensors communicably coupled with the user device.

Statement 12: A method in accordance with any of Statements 1-11, wherein the user device is an output device coupled with a wearable device, the output device is one of a smartphone, a mobile phone, a personal computing device, a laptop computer, a desktop computer, a handheld device, a tablet, a smart vehicle, and combinations thereof.

Statement 13: A method in accordance with any of Statements 1-12, further comprising alerting, via an alert module of the user device, the user when the change in at least one or more of the physiological parameters is detected.

Statement 14: An emotional state monitoring system comprising an emotional state monitoring device comprising one or more physiological sensors operable to be engaged with a body of a user, and a transmitter capable of transmitting and receiving data obtained by the one or more physiological sensors, one or more processors communicatively coupled with the emotional state monitoring device and operable to receive data from the transmitter, the one or more processors having a memory storing instructions thereon, which when executed causes the one or more processors to: obtain, via the one or more physiological sensors, data corresponding to one or more physiological parameters of a user; detect, via at least one of the one or more physiological sensors, a change in at least one of the one or more physiological parameters; request, via the emotional state monitoring device, an emotional state identifier corresponding to the change; and provide, via the emotional state monitoring device, a suggested response based on the emotional state identifier.

Statement 15: A system in accordance with Statement 14, wherein the instructions further cause the one or more processors to generate a database comprising the change in at least one of the one or more physiological parameters and corresponding emotional state identifier; provide a classifier for each the emotional state identifier, the classifier corresponding to an emotion experienced by the user; training the one or more processors to assign the classifier to an subsequent change in at least one of the one or more physiological parameters based at least in part on the database; and requesting, via the user device, a confirmation that the classifier is accurate.

Statement 16: A system in accordance with Statement 14 or Statement 15, wherein when the confirmation is received update the database based on the confirmation.

Statement 17: A system in accordance with any of Statements 14-16, wherein when the request for confirmation is denied request, via the emotional state monitoring device, the emotional state identifier for the subsequent change, and update the database based on the emotional state identifier provided.

Statement 18: A system in accordance with any of Statements 14-17, wherein the instructions further cause the one or more processors to monitor, via the one or more physiological sensors, the one or more physiological parameters to determine if the user complied with the suggested response; determine whether compliance with the suggested response altered the classifier corresponding to the user's emotion; and store, in a second database, the alteration the suggested response had on the user's emotion.

Statement 19: A system in accordance with any of Statements 14-18, wherein the emotional state monitoring device is a wearable device selected from the group comprising a watch, a wrist band, a ring, a necklace, a clothing item, an adhesive patch, a medical device, a hat, a helmet, a band, and combinations thereof.

Statement 20: A system in accordance with any of Statements 14-19, further comprising an output device communicatively coupled with the one or more processors, wherein the output device is one of a smartphone, a mobile phone, a personal computing device, a laptop computer, a desktop computer, a handheld device, a tablet, a smart vehicle, and combinations thereof.

Statement 21: A system in accordance with any of Statements 14-20, wherein the one or more physiological sensors comprises an electrodermal (EDA) sensor, a biomechanical sensor, a galvanic skin response (GSR) sensor, a photoplethysmography (PPG) sensor, an electrocardiogram (EKG) sensor, an inertial measurement sensor, an accelerometer, a gyroscope, a blood pressure sensor, a pulse oximetry (SpO2) sensor, a respiratory rate monitor, a temperature sensor, a humidity sensor, an audio sensor, an air quality sensor, and combinations thereof.

Statement 22: A system in accordance with any of Statements 14-21, wherein at least one of the one or more physiological sensors are coupled with the wearable device.

Statement 23: A system in accordance with any of Statements 14-22, further comprising one or more context sensors coupled communicably coupled with the one or more processors, wherein the one or more context sensors is a smart thermostat, a smart light switch, a smart hub, smart bathroom fixtures, smart microphones, smart refrigerators, vehicles, and/or combinations thereof.

Statement 24: A system in accordance with any of Statements 14-23, wherein the instructions further cause the one or more processors to alert, via an alert module of the user device, the user when the change in at least one or more of the physiological parameters is detected.

Statement 25: An emotional state monitoring device comprising one or more physiological sensors operable to be engaged with a body of a user; one or more processors communicatively coupled with the emotional state monitoring device, the one or more processors having a memory storing instructions thereon, which when executed causes the one or more processors to obtain, via the one or more physiological sensors, data corresponding to one or more physiological parameters of a user; detect, via at least one of the one or more physiological sensors, a change in at least one of the one or more physiological parameters; request, via the emotional state monitoring device, an emotional state identifier corresponding to the change; and provide, via the emotional state monitoring device, a suggested response based on the emotional state identifier.

Statement 26: An emotional state monitoring device in accordance with Statement 25, wherein the one or more physiological sensors comprises an electrodermal (EDA) sensor, a biomechanical sensor, a galvanic skin response (GSR) sensor, a photoplethysmography (PPG) sensor, an electrocardiogram (EKG) sensor, an inertial measurement sensor, an accelerometer, a gyroscope, a blood pressure sensor, a pulse oximetry (SpO2) sensor, a respiratory rate monitor, a temperature sensor, a humidity sensor, an audio sensor, an air quality sensor, and combinations thereof.

Statement 27: An emotional state monitoring device in accordance with Statement 25 or Statement 26, wherein the emotional state monitoring device is a wearable device selected from the group comprising a watch, a wrist band, a ring, a necklace, a clothing item, an adhesive patch, a medical device, a hat, a helmet, a band, and combinations thereof.

Statement 28: An emotional state monitoring device in accordance with Statements 25-27, further comprising one or more context sensors communicative coupled with the one or more processors.

Statement 29: An emotional state monitoring device in accordance with Statements 25-28, wherein the one or more context sensors is a smart thermostat, a smart light switch, a smart hub, smart bathroom fixtures, smart microphones, smart refrigerators, vehicles, and/or combinations thereof.

Statement 30: An emotional state monitoring device in accordance with Statements 25-29, further comprising one or more output device communicatively coupled with the one or more processors, wherein the one or more output device is one of a smartphone, a mobile phone, a personal computing device, a laptop computer, a desktop computer, a handheld device, a tablet, a smart vehicle, and combinations thereof.

Statement 31: An emotional state monitoring device in accordance with Statements 25-31, wherein the instructions further cause the one or more processors to alert, via an alert module of the user device, the user when the change in at least one or more of the physiological parameters is detected.

What is claimed is:

1. A method of monitoring an emotional state, the method comprising:
obtaining, via one or more physiological sensors, data corresponding to one or more physiological parameters of a user;
detecting, via at least one of the one or more physiological sensors, a change in at least one of the one or more physiological parameters;
requesting, via a user device, an emotional state identifier corresponding to the change; and
providing, via the user device, a suggested response based on the emotional state identifier.

2. The method of claim 1, further comprising:
generating, via one or more processors communicatively coupled with the user device, a database comprising the change in the at least one of the one or more physiological parameters and corresponding emotional state identifier; and
providing a classifier for each emotional state identifier, the classifier corresponding to an emotion experienced by the user.

3. The method of claim 2, further comprising:
training the one or more processors to assign the classifier to a subsequent change in at least one of the one or more physiological parameters based at least in part on the database; and
requesting, via the user device, a confirmation that the classifier is accurate.

4. The method of claim 3, wherein when the request for confirmation is received: updating the database based on the confirmation.

5. The method of claim 3, wherein when the request for confirmation is denied,
requesting, via the user device, the emotional state identifier for the subsequent change; and
updating the database based on the emotional state identifier provided.

6. The method of claim 2, further comprising:
monitoring, via the one or more physiological sensors, the one or more physiological parameters to determine if the user complied with the suggested response;
determining, via the one or more processors, whether compliance with the suggested response altered the classifier corresponding to the user's emotion; and
storing, in a second database, the alteration the suggested response had on the user's emotion.

7. The method of claim 1, wherein the one or more physiological sensors comprises an electrodermal (EDA) sensor, a galvanic skin response (GSR) sensor, a photoplethysmography (PPG) sensor, an electrocardiogram (EKG) sensor, an inertial measurement sensor, an accelerometer, a gyroscope, a blood pressure sensor, a pulse oximetry (SpO2) sensor, a respiratory rate monitor, a temperature sensor, a humidity sensor, an audio sensor, an air quality sensor, and combinations thereof.

8. The method of claim 7, wherein at least one of the one or more physiological sensors are coupled with the user device.

9. The method of claim 8, wherein the user device is a wearable device.

10. The method of claim 1, wherein the suggested response comprises a positive emotion reinforcement, a negative emotion mitigation, an emotional state acknowledgement, and combinations thereof.

11. An emotional state monitoring system comprising:
an emotional state monitoring device comprising:
one or more physiological sensors operable to be engaged with a body of a user, and
a transmitter capable of transmitting and receiving data obtained by the one or more physiological sensors,
one or more processors communicatively coupled with the emotional state monitoring device and operable to receive data from the transmitter, the one or more processors having a memory storing instructions thereon, which when executed causes the one or more processors to:
obtain, via the one or more physiological sensors, data corresponding to one or more physiological parameters of a user;
detect, via at least one of the one or more physiological sensors, a change in at least one of the one or more physiological parameters;
request, via the emotional state monitoring device, an emotional state identifier corresponding to the change; and
provide, via the emotional state monitoring device, a suggested response based on the emotional state identifier.

12. The system of claim 11, wherein the instructions further cause the one or more processors to:
generate a database comprising the change in at least one of the one or more physiological parameters and corresponding emotional state identifier;
provide a classifier for each the emotional state identifier, the classifier corresponding to an emotion experienced by the user;
training the one or more processors to assign the classifier to a subsequent change in at least one of the one or more physiological parameters based at least in part on the database; and
requesting, via the user device, a confirmation that the classifier is accurate,
wherein when the confirmation is received:
update the database based on the confirmation, and
wherein when the request for confirmation is denied:
request, via the emotional state monitoring device, the emotional state identifier for the subsequent change, and
update the database based on the emotional state identifier provided.

13. The system of claim 12, wherein the instructions further cause the one or more processors to:
monitor, via the one or more physiological sensors, the one or more physiological parameters to determine if the user complied with the suggested response;
determine whether compliance with the suggested response altered the classifier corresponding to the user's emotion; and
store, in a second database, the alteration the suggested response had on the user's emotion.

14. The system of claim 12, wherein the emotional state monitoring device is a wearable device selected from the group comprising a watch, a wrist band, a ring, a necklace, a clothing item, an adhesive patch, a medical device, a hat, a helmet, a band, and combinations thereof.

15. The system of claim 11, further comprising an output device communicatively coupled with the one or more processors, wherein the output device is one of a smartphone, a mobile phone, a personal computing device, a laptop computer, a desktop computer, a handheld device, a tablet, a smart vehicle, and combinations thereof.

16. The system of claim 11, wherein the one or more physiological sensors comprises an electrodermal (EDA) sensor, a galvanic skin response (GSR) sensor, a photoplethysmography (PPG) sensor, an electrocardiogram (EKG) sensor, an inertial measurement sensor, an accelerometer, a gyroscope, a blood pressure sensor, a pulse oximetry (SpO2) sensor, a respiratory rate monitor, a temperature sensor, a humidity sensor, an audio sensor, an air quality sensor, and combinations thereof.

17. The system of claim 11, further comprising one or more context sensors coupled communicably coupled with the one or more processors.

18. An emotional state monitoring device comprising:
one or more physiological sensors operable to be engaged with a body of a user;
one or more processors communicatively coupled with the emotional state monitoring device, the one or more processors having a memory storing instructions thereon, which when executed causes the one or more processors to:
obtain, via the one or more physiological sensors, data corresponding to one or more physiological parameters of a user;
detect, via at least one of the one or more physiological sensors, a change in at least one of the one or more physiological parameters;
request, via the emotional state monitoring device, an emotional state identifier corresponding to the change; and
provide, via the emotional state monitoring device, a suggested response based on the emotional state identifier.

19. The emotional state monitoring device of claim 18, wherein the one or more physiological sensors comprises an electrodermal (EDA) sensor, a galvanic skin response (GSR) sensor, a photoplethysmography (PPG) sensor, an electrocardiogram (EKG) sensor, an inertial measurement sensor, an accelerometer, a gyroscope, a blood pressure sensor, a pulse oximetry (SpO2) sensor, a respiratory rate monitor, a temperature sensor, a humidity sensor, an audio sensor, an air quality sensor, and combinations thereof.

20. The emotional state monitoring device of claim 18, wherein the emotional state monitoring device is a wearable device selected from the group comprising a watch, a wrist band, a ring, a necklace, a clothing item, an adhesive patch, a medical device, a hat, a helmet, a band, and combinations thereof.
